(19) United States
(12) Patent Application Publication
(10) Pub. No.: US 2010/0022979 A1
(43) Pub. Date: Jan. 28, 2010

(54) DIAPER WITH WASTE RECEPTACLE

(76) Inventors: Kyler Carnegie, Alexandria, VA (US); Colleen A. Hennigan, Alexandria, VA (US)

Correspondence Address: LITMAN LAW OFFICES, LTD., POST OFFICE BOX 15035, CRYSTAL CITY STATION, ARLINGTON, VA, 22215-0035, US

(21) Appl. No.: 11/919,187

(22) PCT Filed: Apr. 26, 2006
(86) PCT No.: PCT/US2006/015900

§ 371 Date: Oct. 24, 2007

Related U.S. Application Data
(60) Provisional application No. 60/674,907, filed on Apr. 26, 2005.

(51) Int. Cl. A61F 13/15

(52) U.S. Cl. 604/385.01

(57) ABSTRACT

The diaper with waste receptacle (10) includes a disposable diaper (14) having an inner surface adapted for contacting the body and an outer surface. A pocket (12) is secured to the outer surface of the diaper (14). An opening of the pocket (12) provides access to the open interior region of the pocket (12). A flexible bag (26) is removably stored within the open interior region. When the diaper (14) is soiled, the user may remove the diaper (14), remove the flexible bag (26) from the pocket (12), and seal and store the soiled diaper (14) within the flexible bag (26) for transport to a waste disposal site. In an alternative embodiment, the flexible bag (126) may be joined directly to the outer surface of the diaper (100) without use of an additional pocket.

### TECHNICAL FIELD

The present invention relates to disposable diapers, and particularly to a diaper with a waste receptacle that provides a disposable diaper in combination with an attached waste bag for enclosing and disposing of the soiled diaper after use.

### BACKGROUND ART

In recent years, cloth diapers have been replaced with disposable diapers, which are made from a variety of economical materials that are not designed for reuse, but which may be disposed of after a single use at a waste disposal site. Disposable diapers typically include adhesive or other non-durable fasteners, allowing the fasteners to be rolled or folded up together with the soiled diaper and carried to a trashcan or other waste collection site. Although easily disposable, the soiled diaper, even in the folded or rolled up state, is still unsanitary and may present a health hazard to those exposed to the excretory waste held therein. Further, soiled diapers are subject to leakage of the excretory waste therein, and the user may become contaminated by the excretory waste during transport thereof to the waste receptacle.

Even in the rolled up or folded up state, a soiled diaper, due to the excretory matter contained therein, generates odoriferous and noxious fumes, and is further a source of bacteria and other harmful microbes to those in the immediate environment. Although sealable containers, such as diaper pails, for collecting soiled diapers for later transport to a waste disposal site are known, these are not easily transportable, particularly when the users are traveling. In addition to the fumes and possibility for airborne infection, the excretory waste matter contained within a folded or rolled-up diaper may leak out of the diaper during transport to the waste site.

Disposable waste bags have been utilized for the storage and transport of soiled diapers, particularly to prevent the transmission of fumes and to separate the soiled diaper from the user's hands during transport. However, such bags are typically provided in packages containing multiple bags, and the bags or packages containing the bags must be transported separately from the diapers. It would be preferable to combine the diaper with a waste storage receptacle as a self-contained unit, which could be manufactured, utilized and transported as a single system, without the need to carry a multiplicity of diaper changing materials.

Thus, a diaper with waste receptacle solving the aforementioned problems is desired.

### DISCLOSURE OF INVENTION

The diaper with waste receptacle includes a diaper for infants, which may be a conventional disposable diaper. It should be understood that the diaper may be used by adults, incontinent persons or any other person desiring a diaper or sanitary garment. A pocket is secured to the outer surface of the diaper, with the pocket being formed with a rear wall and a front wall. The rear wall of the pocket is secured to the outer surface of the diaper. The rear wall and front wall define an open interior region therebetween, with an opening providing access to the open interior region. Preferably, the pocket has a substantially rectangular contour, and is formed from a fluid-impermeable material. The pocket may be secured to either the front or rear portions of the diaper.

A flexible bag is removably stored within the open interior region. The flexible bag is preferably foldable and disposable. When the infant soils the diaper, the user may remove the diaper from the infant, remove the flexible bag from the open interior region, and store the soiled diaper within the flexible bag for transport to a waste disposal site. Optionally, a flap may be provided for selectively covering the opening and securing the flexible bag within the open interior region when not in use. An open end of the flexible bag preferably includes a releasable fastener, allowing the user to seal the soiled diaper within the flexible bag.

Alternatively, the flexible bag may be secured to the diaper without an additional pocket being mounted to the diaper. The flexible bag has an open end and a closed end, and the closed end is secured to the outer surface of the diaper. The flexible bag is folded and the open end is releasably secured to the closed end. When the infant soils the diaper, the diaper may be removed from the infant, and the open end of the flexible bag is detached so that the bag may be unfolded and inverted to wrap around the soiled diaper, and the open end is sealed to enclose the soiled diaper for transport to a waste disposal site.

Although described for use with an infant, the principles of the present invention are also applicable to disposable undergarments, e.g., disposable undergarments for use by individuals suffering from urinary incontinence.

These and other features of the present invention will become readily apparent upon further review of the following specification and drawings.

### BRIEF DESCRIPTION OF THE DRAWINGS

FIG. 1 is an environmental, perspective view of a first embodiment of a diaper with waste receptacle according to the present invention.

FIG. 2 is a plan view of the rear of the diaper with waste receptacle of FIG. 1 according to the present invention.

FIG. 3 is a fragmentary plan view of the rear of the diaper with waste receptacle of FIG. 1 according to the present invention with the pocket broken away.

FIG. 4 is a fragmentary plan view of the embodiment of the diaper with waste receptacle of FIGS. 1-3 with an optionally flap over the pocket, showing the rear of the diaper with the pocket broken away.

FIG. 5 is a plan view of a second embodiment of the diaper with waste receptacle according to the present invention, showing the exterior of the diaper.

FIG. 6 is a partial side view of the embodiment of FIG. 5 in a first, closed configuration.

FIG. 7 is a partial side view of the embodiment of FIG. 5 in a second, partially opened configuration.

FIG. 8 is a partial side view of the embodiment of FIG. 5 in a third, partially opened configuration.

FIG. 9 is a partial side view of the embodiment of FIG. 5 in a fourth, open configuration.

Similar reference characters denote corresponding features consistently throughout the attached drawings.

### BEST MODES FOR CARRYING OUT THE INVENTION

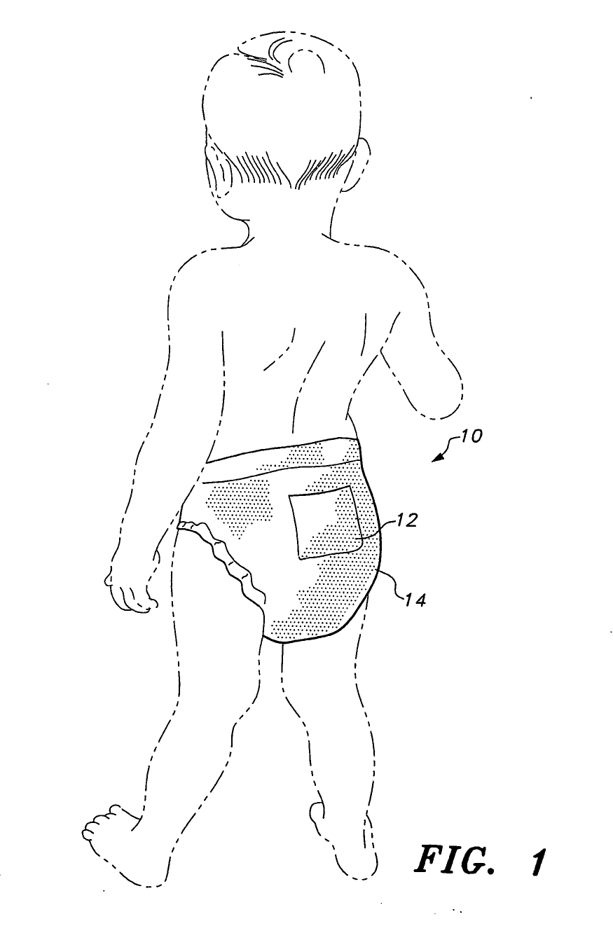

FIGS. 1-3 show a first embodiment of a diaper with waste receptacle, designated generally as 10 in the drawings, which includes a disposable diaper 14 having a pocket 12 mounted thereon. A flexible bag 26 for disposing of the soiled diaper 14 after use is preferably folded and stored within the pocket 12. Diaper 14 is a conventional disposable diaper. The diaper is non-washable and is not reusable, being made from polymeric materials having an outer surface (which may be made from a fluid impermeable material) and an absorbent inner surface adapted for contacting the body of a wearer, as known in the art. Diaper 14 may include releasable fasteners, such as adhesive tabs or hook-and-loop type fasteners, as are commonly applied to disposable diapers. It should be understood that, although diaper 14 is shown being worn by an infant in FIG. 1, diaper 14 may also be a sanitary garment or disposable undergarment worn by older children or adults, e.g., for urinary incontinence. Diaper 14 includes a front portion 18, a rear portion 16, and a central portion 20. Elastic gathers or strips 22 may be secured to outer edges of the central portion 20 in order to form a snug fit about the wearer's legs. The disposable diaper 14 may also have an elastic waistband. However, while the present invention has been described with reference to preferred embodiments having multilayer construction, integral releasable fasteners, elastic leg gathers and elastic waistbands, it will be understood that the scope of the present invention extends to a disposable diaper 14 of any construction that is not washable, not reusable, and designed to be disposed of after a single use.

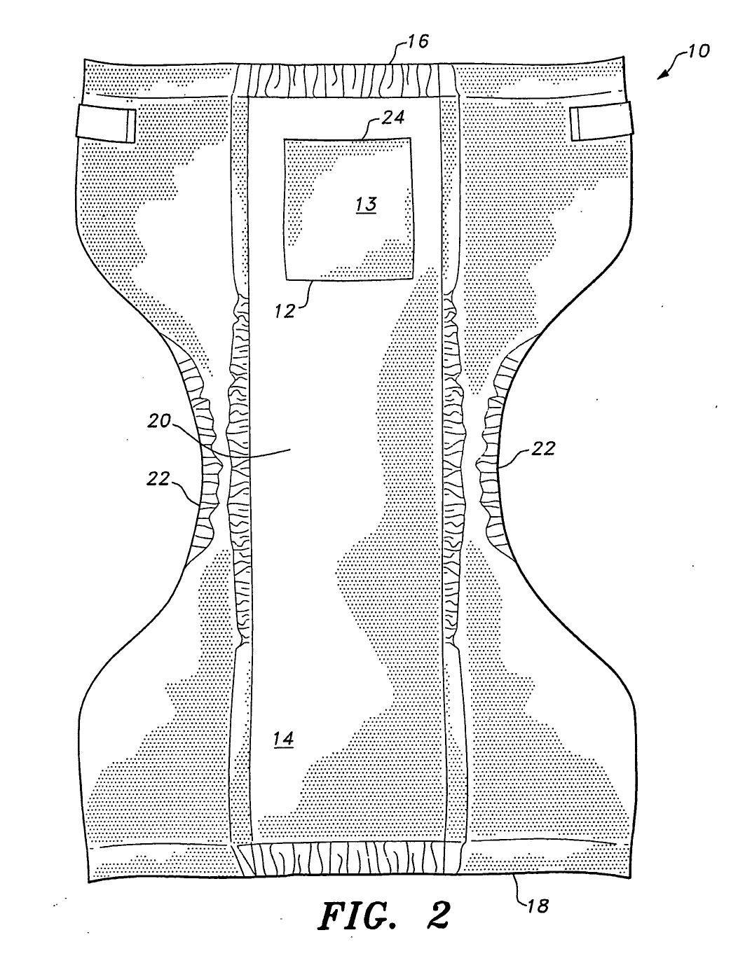

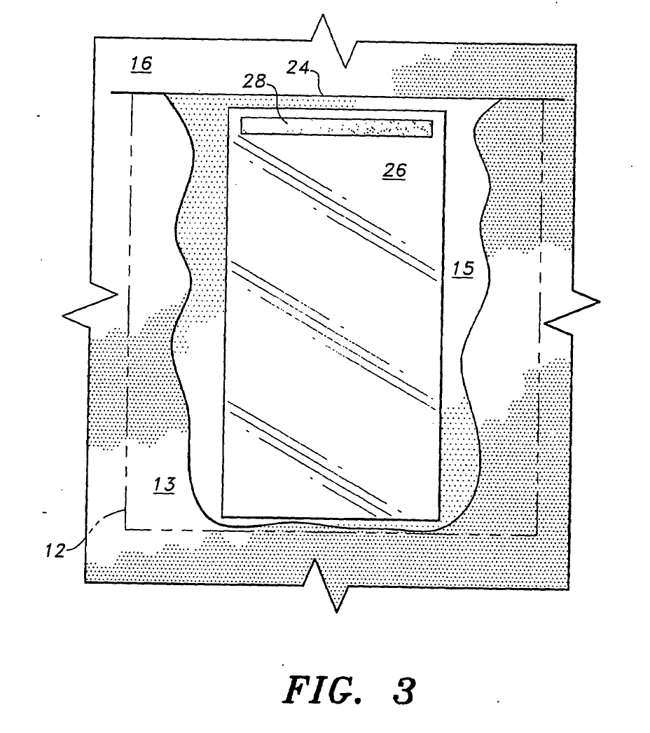

As best shown in FIGS. 2 and 3, pocket 12 has a front wall 13 and a rear wall 15 defining an open interior region therebetween. Pocket 12 may be formed from a single sheet folded in half and joined along lateral edges, or by two separate sheets joined along bottom and side edges. An outer surface of the rear wall 15 is irremovably attached to the outer surface of diaper 14 by adhesive bonding, heat sealing, ultrasonic welding, stitching, or any other method known in the art. Pocket 12, or at least rear wall 15, is preferably formed from a fluid-impermeable material, such as flexible plastic film, or any other suitable material that will prevent the leakage of bodily fluids from within diaper 14 to enter the interior of pocket 12, where urine absorbed from the diaper might contaminate flexible bag 26 with moisture, bacteria, etc. The material forming pocket 12 is further preferably resistant to the transmission of bacteria and other microbes therethrough, which may be contained in the child's waste. Although shown as having a substantially rectangular contour in the drawings, it should be understood that pocket 12 may have any suitable shape or size. In the preferred embodiment, pocket 12 has a substantially square contour, with an upper edge, a lower edge and a pair of opposed side edges. The lower edges and opposed side edges of front wall 13 and rear wall 15 are attached or bonded to one another, respectively, with the upper edges defining opening 24 providing access to the open interior region of the pocket 12.

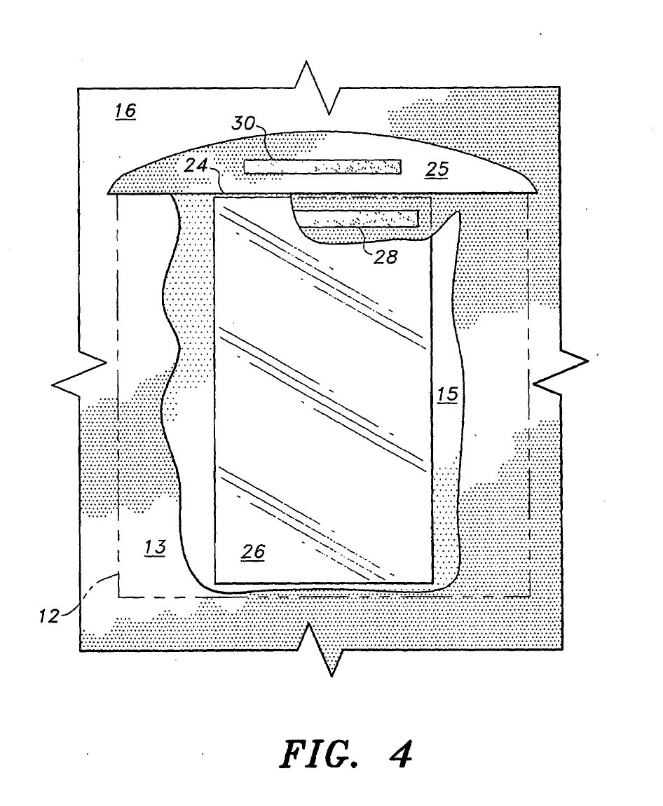

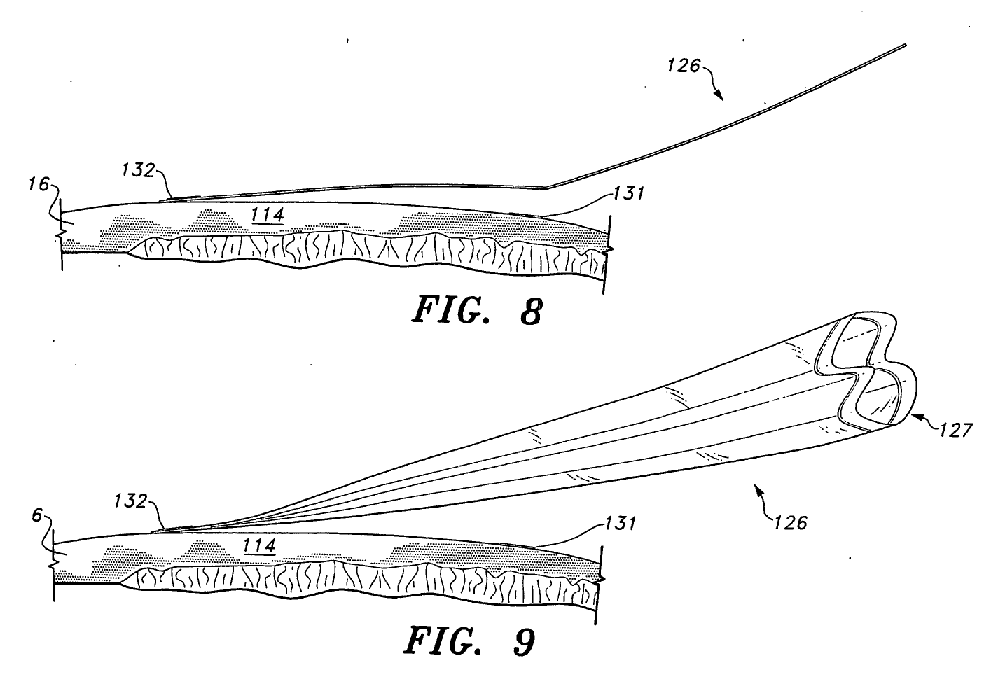

Flexible bag 26 is stored within the pocket 12. The flexible bag 26 may be formed from flexible plastic film or any other suitable material for the sanitary disposal of a soiled diaper. Flexible bag 26 is preferably made from fluid impermeable material so that moisture and odors from the soiled diaper are contained within the bag after sealing. Flexible bag 26 may include a selectively activated seal or fastener, such as a bead-and-groove fastener or Ziploc fastener, for example, allowing the user to seal a soiled diaper within the bag 26 for storage and transport (shown, and described in further detail below, in the embodiment of FIG. 9). As illustrated in FIG. 3, a releasable fastener 28, which may be adhesive tape, a hook and loop-type fastener, or any other suitable releasable fastener, is provided for releasably securing the flexible bag 26 within the pocket when not in use. The releasable fastener 28 may be releasably mounted on the inner surface of front wall 13 or, alternatively, may be releasably mounted on the inner surface of rear wall 15 (shown in FIG. 4). Although shown as being positioned adjacent opening 24, it should be understood that fastener 28 may be secured to any desired position within pocket 12.

Although shown in FIGS. 1 and 2 as being mounted on the rear portion 16 of diaper 14, it should be understood that pocket 12 may be mounted on the front portion 18, or on any other suitable region, of the diaper 14. Pocket 12 is preferably mounted on rear portion 16 to prevent the infant from accidentally removing bag 26 from the pocket.

Further, as shown in FIG. 4, a flap 25 may optionally be mounted to the outer surface of diaper 14, positioned above the opening 24 of pocket 12 in order to releasably cover and seal opening 24. Flap 25 is provided with a releasable fastener 30, similar to releasable fastener 28, which may be adhesive tape, a hook and loop-type fastener, or any other suitable releasable fastener for releasably joining flap 25 to the outer surface of front sheet 13.

In use, a soiled diaper 14 is removed from the infant and the flexible bag 26 is removed from pocket 12 for the storage of the soiled diaper 14 therein. The flexible bag 26 is sealed, allowing the user to transport the diaper 14 to a waste receptacle without leakage of excretory waste, and sealing the odor of a smelly diaper within the bag 26 so that the odor is not released into the ambient environment.

In the alternative embodiment illustrated in FIG. 5, the diaper with waste receptacle, designated generally as 100 in the drawings, includes a disposable diaper 114 similar to disposable diaper 14 of FIGS. 1-4. Diaper 114 includes a rear portion 116, a central portion 120 and elastic strips 122, similar to those described above with respect to the embodiment shown in FIG. 2. Unlike the embodiments of FIGS. 1-4, however, the diaper with waste receptacle 100 includes a flexible bag 126 that is secured to the outer surface of diaper 114 without an additional pocket. Flexible bag 126 is preferably formed from similar materials as those described above with respect to flexible bag 26.

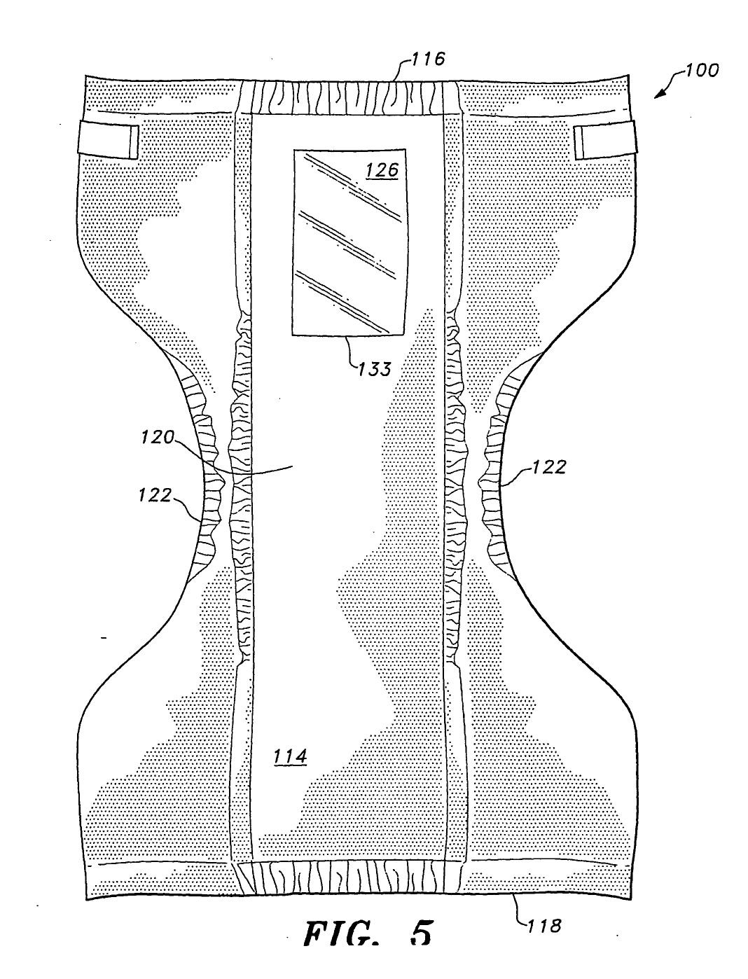

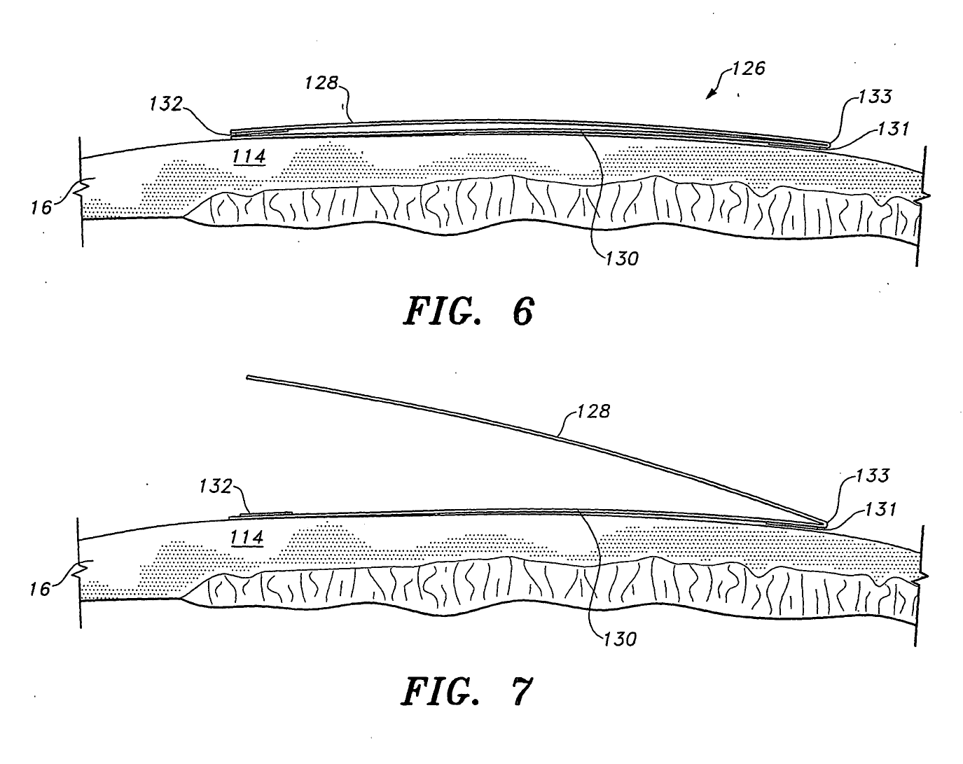

The flexible bag 126, shown in FIG. 6 in its folded and closed configuration when not in use, includes a lower bag portion 130, which is positioned adjacent the outer surface of rear portion 116 of diaper 114, and an upper bag portion 128. Flexible bag 126 is preferably folded, approximately halfway through its length, as indicated by the fold at 133. As described above with reference to the embodiments of FIGS. 1-4, the flexible bag 126 may be positioned on any suitable location of diaper 114. However, in the preferred embodiment, bag 126 is mounted on rear portion 116 to prevent the infant from accidentally removing and opening the bag, which could create a safety hazard for the infant.

Flexible bag 126 has an open end and a closed end. The closed end of flexible bag 126 is fixed to the outer surface of rear portion 116, shown in FIG. 6 as being the left edge of lower bag portion 130. A releasable fastener 131, similar to releasable fasteners 28, 30, described above, releasably secures the opposed end of lower portion 130 to the outer surface of the diaper. Releasable fastener 131 is positioned adjacent folded edge 133 of bag 126. The open end of flexible bag 126 is releasably secured to the closed end by a second releasable fastener 132, similar to releasable fasteners 28, 30, 131, described above.

Flexible bag 126 preferably includes perforated side edges and a perforated lower edge. Prior to the unfolding of the flexible bag 126, as illustrated in FIG. 7, the main body portion of flexible bag 126 is torn away, via the perforations, from the lower edge and side edges. Thus, flexible bag 126 is prevented from accidental unfolding, which may be hazardous to the child.

In the partially open configuration of FIG. 7, the open end of bag 126 (shown in the drawing as the left edge of upper portion 128) has been separated from the closed end (shown in the drawing as the left edge of lower portion 130) through the disengagement of releasable fastener 132. In the second partially open configuration of FIG. 8, releasable fastener 131 has been disengaged to separate the folded end of bag 126 from diaper 114. Shown in FIG. 8, bag 126 is further unfolded and extended to its full length.

In the open configuration shown in FIG. 9, the open end of flexible bag 126 is fully opened by the user so that flexible bag 126 may be inverted and wrapped around the soiled diaper for later transport to a waste disposal site. Alternatively, the closed end of bag 126 may be releasably secured to the diaper 114, allowing the user to separate the bag 126 from the diaper 114 for storage of the soiled diaper in the bag 126.

Further, as shown in FIG. 9, the open end of flexible bag 126 may have a releasable fastener, such as the Ziploc fastener shown, 127 secured thereto, allowing the user to seal the soiled diaper within the flexible bag 126 for later transport. The releasable fastener 127 may be any suitable fastener that will prevent the leakage of fluids or odor from within the bag when a soiled diaper is contained within flexible bag 126.

Further, flexible bag 126 may be scented, or contain a scented material or scented layer therein, for masking the scent of the soiled diaper stored therein. Additionally, the flexible bag 126 may be provided with a particular color or distinctive indicia associated with a particularly sized diaper, to which the flexible bag 126 is attached.

It is to be understood that the present invention is not limited to the embodiments described above, but encompasses any and all embodiments within the scope of the following claims.

We claim:

1. A diaper with waste receptacle, comprising:
a disposable diaper having a front portion, a rear portion and a central portion, the diaper having an inner surface adapted for contacting a human body, and an outer surface;
a pocket disposed on the outer surface of the diaper, the pocket having a rear wall attached to the outer surface of the diaper and a front wall attached to the rear wall, the front and rear walls defining an opening to an open interior region therebetween; and
a flexible bag removably stored within the open interior region, the bag being dimensioned and configured for enclosing the disposable diaper therein;
whereby when the diaper is soiled, the user may remove the diaper from the human body and store the soiled diaper within the flexible bag for transport to a waste disposal site.

2. The diaper with waste receptacle as recited in claim 1, wherein said pocket is formed from two separate sheets joined each other along respective lower edges thereof, and along respective opposed side edges thereof.

3. The diaper with waste receptacle as recited in claim 2, wherein said pocket has a substantially rectangular contour.

4. The diaper with waste receptacle according to claim 1, wherein said pocket is made from fluid impermeable material in order to prevent said flexible bag from contamination when the diaper is soiled while the flexible bag is stored in said pocket.

5. The diaper with waste receptacle as recited in claim 1, further comprising means for releasably securing said flexible bag to an inner surface of said front wall when said flexible bag is stored within the open interior region.

6. The diaper with waste receptacle as recited in claim 1, further comprising means for releasably securing said flexible bag to an inner surface of said rear wall when said flexible bag is stored within the open interior region.

7. The diaper with waste receptacle as recited in claim 1, further comprising a flap mounted on the outer surface of said diaper adjacent the opening of said pocket for releasably sealing and covering said opening.

8. The diaper with waste receptacle as recited in claim 7, further comprising means for releasably securing said flap to an outer surface of said front wall.

9. The diaper with waste receptacle as recited in claim 1, wherein said pocket is mounted on the front portion of said diaper.

10. The diaper with waste receptacle as recited in claim 1, wherein said pocket is mounted on the rear portion of said diaper.

11. The diaper with waste receptacle as recited in claim 1, further comprising means for selectively sealing an open end of said flexible bag.

12. A diaper with waste receptacle, comprising:
a disposable diaper having a front portion, a rear portion and a central portion, the diaper having an inner surface adapted for contacting a human body, and an outer surface;
a flexible bag having an open end and a closed end, the closed end being secured to the outer surface of the diaper, the bag being dimensioned and configured for enclosing the disposable diaper;
whereby when the diaper is soiled, the user may remove the diaper from the human body and store soiled diaper within the flexible bag for transport to a waste disposal site.

13. The diaper with waste receptacle as recited in claim 12, further comprising means for releasably attaching the open end of said flexible bag to the closed end, said flexible bag being folded to form a closed configuration until said bag is needed to enclose the diaper.

14. The diaper with waste receptacle as recited in claim 13, wherein the fold in said bag is releasably attached to the outer surface of said diaper.

15. The diaper with waste receptacle as recited in claim 12, wherein said flexible bag is attached to the front portion of said diaper.

16. The diaper with waste receptacle as recited in claim 12, wherein said flexible bag is attached to the rear portion of said diaper.

17. The diaper with waste receptacle as recited in claim 12, further comprising means for selectively sealing the open end of said flexible bag.

18. The diaper with waste receptacle according to claim 12, wherein the closed end of said flexible bag is permanently attached to the outer surface of said diaper, said flexible bag being inverted to enclose said diaper when said diaper is soiled.

19. The diaper with waste receptacle according to claim 12, wherein the closed end of said flexible bag is removably attached to said diaper, said flexible bag being detached from said diaper in order to enclose said diaper when said diaper is soiled.
